(19) United States
(12) Patent Application Publication
(10) Pub. No.: US 2012/0202239 A1
(43) Pub. Date: Aug. 9, 2012

(54) MATERIALS, MONITORING, AND CONTROLLING TISSUE GROWTH USING MAGNETIC NANOPARTICLES

(76) Inventor: Ezekiel Kruglick, Poway, CA (US)

(21) Appl. No.: 13/386,899

(22) PCT Filed: Feb. 4, 2011
(86) PCT No.: PCT/US2010/023700

§ 371 Date: Jan. 24, 2012

(51) Int. Cl. G01N 27/72; C12M 1/34; C12N 5/071; C12N 13/00; B82Y 5/00

(52) U.S. Cl. 435/29; 435/395; 435/173.1; 435/287.1; 977/838; 977/960

(57) ABSTRACT

Systems and method for releasing a biological factor in a tissue or organ are disclosed. The system includes one or more nanoparticles distributed in the tissue or organ, the nanoparticles including the biological factor; and a magnetic field generator configured to generate a magnetic field at a first frequency and to apply to the tissue or organ the magnetic field at the first frequency thereby causing at least some of the biological factor to be released from each of the nanoparticles into the tissue or organ.

### BACKGROUND

Unless otherwise indicated herein, the materials described in this section are not prior art to the claims in this application and are not admitted to be prior art by inclusion in this section.

Nanocarriers may be used in biological applications to provide controlled drug distribution. For instance, nanoparticle spheres having a drug have been used to distribute drugs in a controlled manner. The nanoparticle spheres having the drug carry the drug to a particular location requiring treatment. Once the nanoparticle spheres have been distributed to the particular location, external triggers may be used to cause the nanoparticle spheres to release the drug.

### SUMMARY

Techniques are generally described that include scaffolds, engineered tissues, systems, and/or methods.

The present disclosure describes scaffolds. Examples of scaffolds may include a scaffold material and at least one magnetic nanoparticle. The scaffold material may be configured to provide structural support for growth of at least one cell. The magnetic nanoparticle may be supported by the material and may be configured to rupture in response to a magnetic field.

The present disclosure describes engineered tissues. Examples of engineered tissues may include a scaffold and at least one cell. The scaffold may include a scaffold material The at least one cell may be at least partially supported by the scaffold material, and may include at least one magnetic nanoparticle. The at least one magnetic nanoparticle may be configured to rupture in response to a magnetic field.

The present disclosure describes methods of evaluating engineered tissue. Example methods may include providing an engineered tissue, and generating a magnetic resonance image of the engineered tissue. The engineered tissue may include a scaffold and at least one cell at least partially supported by the scaffold. The scaffold may include a scaffold material. At least one of the at least one cell or the scaffold may include at least one magnetic nanoparticle.

The present disclosure describes methods of controlling tissue growth. Example methods may include applying a magnetic field to an engineered tissue, rupturing at least one magnetic nanoparticle, and releasing a biological factor from the at least one magnetic nanoparticle. The engineered tissue may include a scaffold and at least one cell at least partially supported by the scaffold. The scaffold may include a scaffold material. The at least one cell or the scaffold may include at least one magnetic nanoparticle.

The present disclosure describes magnetic resonance imaging systems. Example systems may include a magnetic field generator and a controller. The controller may be coupled to the magnetic field generator and configured to provide at least one first control signal to the magnetic field generator to generate a first magnetic field having a first frequency configured to generate an image of an engineered tissue. The controller may be further configured to provide at least one second control signal to the magnetic field generator to generate a second magnetic field having a second frequency configured to rupture at least one magnetic nanoparticle associated with the engineered tissue.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several examples in accordance with the disclosure and are, therefore, not to be considered limiting of its scope, the disclosure will be described with additional specificity and detail through use of the accompanying drawings.

FIG. 1 is a schematic illustration of a cross-section of a scaffold.

FIGS. 2A-2C are schematic illustrations of a magnetic nanoparticle 100.

FIG. 3 is a schematic illustration of an embodiment of an engineered tissue 200 having a plurality of magnetic nanoparticles 100.

FIG. 4 is a flowchart illustrating a method 400 of evaluating engineered tissue.

FIG. 5 is a flowchart of a method 500 of controlling tissue growth.

FIG. 6 is a schematic illustration of a magnetic resonance imaging system 600.

### DETAILED DESCRIPTION

The following detailed description, reference is made to the accompanying drawings, which form a part hereof. In the drawings, similar symbols typically identify similar components, unless context dictates otherwise. The illustrative examples described in the detailed description, drawings, and claims are not meant to be limiting. Other examples may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are implicitly contemplated herein.

This disclosure is drawn, inter alia, to methods, systems, devices, and/or apparatus generally related to the use of magnetic nanoparticles. The magnetic nanoparticles may be supported by a scaffold material or incorporated in a cell at least partially supported by a scaffold. The magnetic nanoparticle may be configured to rupture in response to a magnetic field. As will be described further below, magnetic particles may in some examples facilitate and/or enhance imaging of an engineered tissue and/or the release of a biological factor or other analyte.

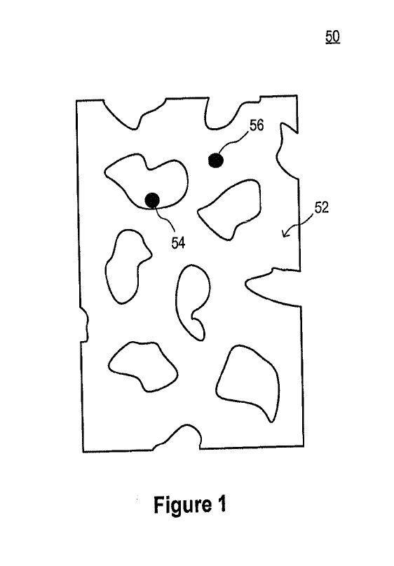

FIG. 1 is a schematic illustration of a cross-section of a scaffold. The scaffold 50 may include a scaffold material 52. The scaffold 50 may also include magnetic nanoparticles 54 and 56.

The scaffold material 52 may be implemented using any scaffold appropriate for tissue engineering. Examples include, but are not limited to, polymers and hydrogels. Other materials that may be used to implement the scaffold material 52 may include agar, polyesters or collagen. The material may be porous and may define any number and size of pores. The scaffold material 52 may be configured to provide structural support for growth of at least one cell. Accordingly, one or more cells, which may form all or part of a tissue or organ, may be grown on the scaffold 50.

Any cells adapted for growth on scaffolds may be grown on the scaffold 50 including, but not limited to, gland cells, hormone secreting cells, epithelial cells, hepatocytes, adipocytes, kidney cells, pancreatic cells, blood cells, immune system cells, pigment cells, germ cells, stem cells, neural cells, and muscle cells (including myocardial cells). In this manner, substantially any tissue and/or all or a portion of any organ may be grown on the scaffold 50. Examples of tissues which may be grown include connective tissue, epithelial tissue, bone, cartilage, fat, blood vessel, muscle, and nerve tissue. Examples of organs which may be grown accordingly include, but are not limited to, bladder, skin, liver, and pancreas. When stem cells are used, the stem cells may be controlled to differentiate into predetermined cell types. Examples of the invention may be used as a tool for any form of tissue engineering for any cell, tissue, and/or organ.

The scaffold 50 may include one or more magnetic nanoparticles, such as the magnetic nanoparticles 54 and 56 shown in FIG. 1, The magnetic nanoparticles include one or more magnetic materials, such as but not limited to iron oxide, paramagnetic materials, or ferromagnetic materials. The magnetic nanoparticles further may have a dimension on the order of micrometers or nanometers. For example, examples of magnetic nanoparticles described herein may include particles (such as spheres) having a diameter (or one dimension) equal to or less than 500 μm, equal to or less than 250 μm, equal to or less than 100 μm, equal to or less than 50 μm, equal to or less than 100 nm, equal to or less than 80 nm in some examples, equal to or less than 60 nm in some examples, equal to or less than 40 nm in some examples, equal to or less than 20 nm in some examples, and equal to or less than 10 nm in some examples. In one or more of these embodiments, a lower bound of the diameter (or one dimension) may include but is not limited to 250 μm, 100 μm, 50 μm, 5 μm, 100 nm, 80 nm, 60 nm, 40 nm, 20 nm or 10 nm. Generally, the larger the diameter of the magnetic nanoparticle, the lower the frequency of the magnetic field which may be needed to stimulate the magnetic nanoparticle.

Examples of magnetic nanoparticles described herein may include a magnetic shell of a magnetic material, such as but not limited to superparamagnetic magnetic material such as iron oxide. The thickness of the shell may generally vary according to the particular process used to make the magnetic nanoparticle. The shell may enclose a core which may be hollow or made of another material, such as but not limited to silica, polymer such as polyvinyl pyrrolidone (PVP), calcium, lipid, or combinations thereof. Examples of suitable magnetic particles having a polymer-modified silica core and magnetic shell are described in Hu, et. al. “Core/Single-crystal-shell nanospheres for controlled drug release via a magnetically triggered rupturing mechanism,” Adv. Mater. 2008, 9999, p. 1-6, which article is hereby incorporated by reference in its entirety for any purpose.

The core of example magnetic nanoparticles described herein may include a biological factor or other chemical. The shell of magnetic nanoparticles described herein may be configured to rupture responsive to application of a magnetic field, releasing the biological factor or other chemical contained in the core. Examples of chemicals which may be contained in the core include but are not limited to growth factors, amino acids, inhibitors, drugs, toxins, and combinations thereof. Examples of growth factors include, but are not limited to, autocrine motility factor, bone morphogenic proteins, epidermal growth factor, erythropoietin, fibroblast growth factor, granulocyte-colony stimulating factor, granulocyte-macrophage colony stimulating factor, growth differentiation factor-9, hepatocyte growth factor, hepatoma derived growth factor, insulin-like growth factor, myostatin, nerve growth factor, platelet-derived growth factor, thrombopoietin, transforming growth factor alpha, transforming growth factor beta, vascular endothelial growth factor, and placental growth factor.

Examples of amino acids include, but are not limited to, isoleucine, leucine, lysine, methionine, phenylalanine, threonine, tryptophan, valine, alanine, asparagine, aspartic acid, cysteine, glutamic acid, glutamine, glycine, proline, selenocysteine, serine, tyosine, arginine, histidine, ornithine, and taurine.

Examples of inhibitors include, but are not limited to, protease inhibitors, protein kinase inhibitors, diisopropylfiuorophosphate, and α-difluoromethylornithine.

The magnetic nanoparticles such as the nanoparticle 54 and 56 may be supported by the scaffold material 52, attached to the scaffold material 52, embedded in the scaffold material 52, may be contained in cells supported by the scaffold material 52, or combinations thereof. The magnetic nanoparticles may be physically mixed with the scaffold material 52 to incorporate the nanoparticles into the scaffold. For example, the nanoparticles may be mixed with an agar or hydrogel precursor used to form the scaffold. The nanoparticles may alternatively or in addition be patterned into the material, such as by injection using a gas gun. The nanoparticles may be injected into cells mixed into or supported by the scaffold material. For example, nanoparticles may be injected into cells using a gas gun and the nanoparticle-containing cells may be mixed with agar or hydrogel precursor used to form the scaffold. In some examples, the scaffold may be formed using layers of scaffold material, and different cells and/or different nanoparticles may be included in the different layers to facilitate the growth of a particular cell or organ. As will be described further below, the magnetic nanoparticles 54 and 56 may be configured to rupture responsive to a magnetic field, and accordingly may release chemical stored therein, affecting cell growth.

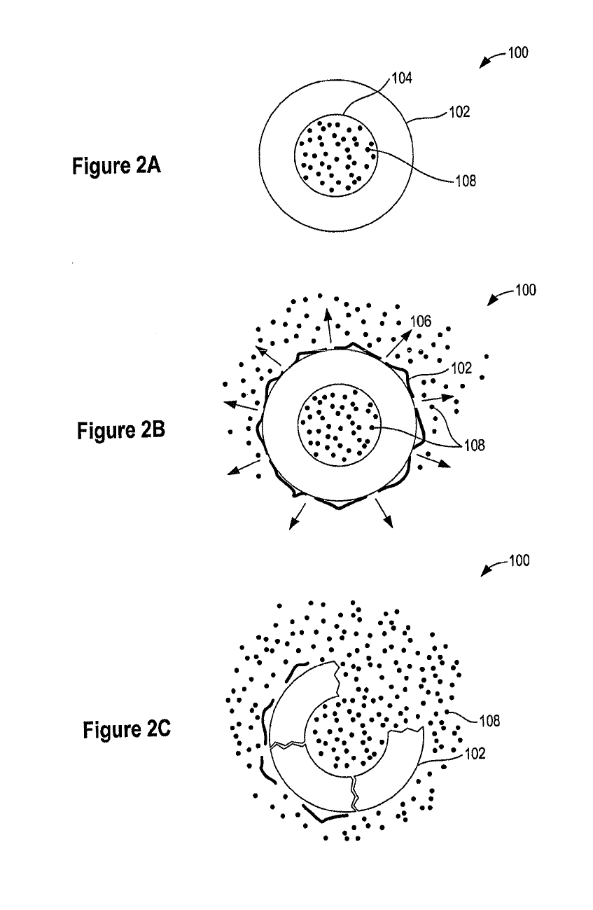

FIGS. 2A-2C are schematic illustrations of a magnetic nanoparticle 100. The magnetic nanoparticle 100 may be used to implement the nanoparticles 54 or 56 of FIG. 1 in some examples. FIG. 2A is a schematic illustration of the magnetic nanoparticle 100 having an outer shell 102 and an inner core 104, as generally discussed above. The inner core 104 may generally include any chemical 108 able to be retained within the outer shell 102 of the magnetic nanoparticle 100.

As will be described further below, exposure to a magnetic field may disrupt a structure of the outer shell 102. FIG. 2B is a schematic illustration of the magnetic nanoparticle 100 having a reversibly ruptured outer shell 102. A magnetic field may generate a force illustrated by the arrows in FIG. 2B. Disruption of the molecular structure of the magnetic shell 102 may accordingly result in rupture of the outer shell 102 and chemical 108 being released from the core 104. For example, the outer shell 102 may become disrupted following exposure to a magnetic field at a particular frequency for a period of time sufficient to disrupt the structure of the shell 102 but insufficient to irreversibly rupture the shell 102. In response to the exposure to the magnetic field, vibrations caused by the magnetic field may cause reversible deformation in the outer shell 102. The vibration may enlarge nanometer cracks 106 in the outer shell 102 and allow a chemical in the inner core 104 to escape from the magnetic nanoparticle 100. When the magnetic nanoparticle 100 is no longer exposed to the magnetic field, the vibration may stop, allowing the enlarged nanometer cracks to settle. In some examples, the cracks may settle to a state that prevents the biological factor in the inner core 104 from escaping through the outer shell 102. In this manner, rupture of magnetic nanoparticles may be reversible.

FIG. 2C is a schematic illustration of the magnetic nanoparticle 100 after being ruptured responsive to a magnetic field. In FIG. 2C, damage to the right side of the outer shell 102 may be irreversible. This irreversible damage to the outer shell 102 may cause some or all of the chemical in the inner core 104 to be released from the outer shell 102. Thus, in this example even when the magnetic nanoparticle 100 is no longer exposed to the magnetic field, the chemical may continue to be released from the magnetic nanoparticle 100.

Accordingly, magnetic nanoparticles described herein may allow chemicals contained in the magnetic nanoparticles to pass into the surrounding environment responsive to a magnetic field. This may allow for a controlled release of a chemical into a particular region of an engineered tissue, as will be described further below.

The size of the magnetic nanoparticle 100 and/or the thickness of the outer shell 102 may affect the length of time that the magnetic nanoparticle 100 may be exposed to a magnetic field before reversibly or irreversibly rupturing. That is, as the diameter of the nanoparticle sphere or the thickness of the outer shell 102 increases, the duration of magnetic field exposure necessary to rupture the magnetic nanoparticle may increase. The time the nanoparticle spheres 100 are exposed to the magnetic field may vary. In general, the exposure time may be any time that would excite the outer shell 102 to allow at least some of the biological factor to escape. In some examples, the exposure time may be on the order of seconds to reversibly rupture a magnetic nanoparticle, and on the order of minutes to irreversibly rupture a magnetic nanoparticle. In some examples, exposure to reversibly rupture a magnetic nanoparticle may range from 2 seconds to 5 seconds, 1 second to 10 seconds in some examples, 1 second to 30 seconds in some examples. In some examples, exposure time to irreversibly rupture a magnetic nanoparticle may range from 1 minute to 5 minutes, 1 minute to 3 minutes in some examples, and 1 minute to 2 minutes in other examples. Other exposure times may be used. In some examples, exposure times may be up to 20 minutes.

The size of the nanoparticle sphere 100 and/or the thickness of the outer shell 102 may also affect the frequency of magnetic field which may rupture the magnetic nanoparticle. In some examples, the magnetic field frequency used to rupture magnetic nanoparticles may be approximately between 10-100 kHz, and in some examples the frequency used to rupture the magnetic nanoparticles may be between 50-100 kHz. For example, smaller magnetic spheres 100 and/or thinner outer shells 102 may require a higher frequency magnetic field to rupture the magnetic nanoparticle 100. For larger magnetic nanoparticles 100 or thicker outer shells 102 a lower frequency may be used to disrupt the magnetic nanoparticle 100. In some examples, the magnetic nanoparticle 100 may have a diameter of approximately 10-50 nanometers. In some examples, the thickness of the outer shell 102 may be between approximately 1-15 nanometers, between approximately 5-10 nanometers in some examples, and between approximately 1-5 nanometers in some examples. However, any diameter or thickness may be used that may rupture responsive to an applied magnetic field and contain a chemical within the magnetic nanoparticle.

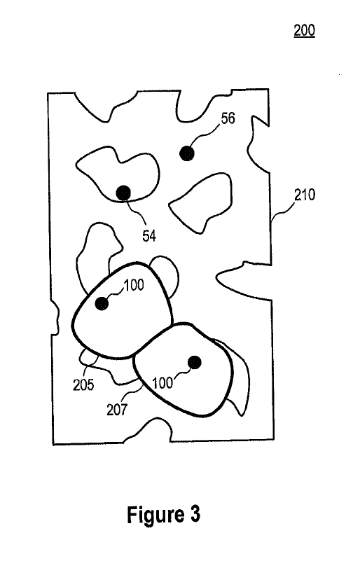

FIG. 3 is a schematic illustration of an embodiment of an engineered tissue 200 having a plurality of magnetic nanoparticles 100. The engineered tissue 200 includes a plurality of cells such as cells 205 and 207 growing on a scaffold 210.

The magnetic nanoparticles 100 may be included in the scaffold 210, one or more of the cells 205 or 207, or both the scaffold 210 and one or more of the cells 205 or 207. In some examples, magnetic nanoparticles may be mixed or introduced onto or into the cells 205 or 207 prior to introducing the cells 205 or 207 to the scaffold 210. The magnetic nanoparticles 100 may be injected into scaffold 210 after formation of the scaffold 210. The magnetic nanoparticles 100 may alternatively or in addition be mixed with materials used to form the scaffold 210, such as agar or hydrogel precursor. The magnetic nanoparticles 100 may alternatively or in addition be injected into cells that are mixed with the scaffold material or supported by the scaffold 210. Cells may be cultured in a nanoparticle-rich environment, generating cells containing the nanoparticles.

The location of the magnetic nanoparticles 100 in the scaffold and/or cells may determine where chemical may be released and introduced to a growing engineered tissue. The placement of magnetic nanoparticles 100 may be controlled through use of a gas gun to inject the particles. In some examples, the scaffold may be formed by layering material layers. Different nanoparticles may be mixed in the materials used to form each layer, resulting in some control of the patterned scaffold. Examples of suitable cells and tissues have been described above.

The nanoparticles may be uniform in size, or have different sizes. For example, the scaffold may have one size of nanoparticle introduced before the cells, such as by mixing the nanoparticle into materials used to form the scaffold or injecting the nanoparticles with a gas gun. The cells themselves may contain a different size nanoparticle, incorporated into the cell by gas gun injection or culture in a nanoparticle rich environment, for example. The two nanoparticle types may be responsive to different frequencies and/or exposure times. This may allow differential or compound biological signals to be triggered. A first exposure time and/or frequency may rupture the first size nanoparticles, and then a subsequent second exposure time and/or frequency may rupture the second size nanoparticles.

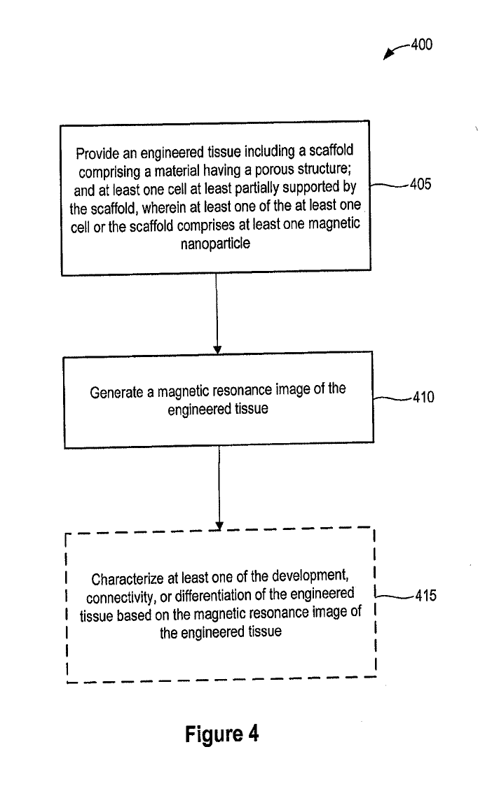

FIG. 4 is a flowchart illustrating a method 400 of evaluating engineered tissue. In block 405, an engineered tissue may be provided. The engineered tissue may include a scaffold comprising a scaffold material and a cell supported by the scaffold. At least one of the cell or the scaffold may include at least one magnetic nanoparticle. Suitable examples of engineered tissues have been described above, and include the engineered tissue 200 of FIG. 3.

Block 405 may be followed by block 410. In block 410, a magnetic resonance image (MRI) of the engineered tissue may be generated. Substantially any MRI system may be used to generate an image of the engineered tissue, and example systems will be described further below. In some examples, the magnetic resonance image generated may be a diffusion spectrum image. The diffusion spectrum image may be generated in part by measuring a water diffusion density spectra associated with regions or cells of the engineered tissue. A diffusion spectrum image may map a diffusion tensor of water associated with at least one cell in the engineered tissue. Generally, water may diffuse at a faster rate in a direction aligned with structure of the engineered tissue, and more slowly in a direction perpendicular to the structure. A diffusion spectrum image may be based, at least in part, on a water diffusion rate and/or direction for each image region, such as a pixel. In this manner, diffusion spectrum MRI images may advantageously illustrate structure of an engineered tissue.

The inclusion of magnetic nanoparticles in the engineered tissue provided in block 405 may advantageously increase the contrast of the MRI image generated in the block 410.

Block 415 may follow block 410. In block 415, an engineered tissue may be evaluated by characterizing at least one of development, connectivity, or differentiation of the engineered tissue based on the magnetic resonance image of the engineered tissue. The evaluation may be performed by a human observer of the MRI image. The evaluation may in some examples be performed by a software process analyzing the MRI image data. The nanoparticles may serve has a contrast enhancer. Diffusion MRI images allow for observation of interconnections between structures such as, but not limited to, tissues, blood vessels, and striations of muscle tissue. Observation of these structures allows an assessment of the development, connectivity, or differentiation of the tissue.

Accordingly, an MRI may be used to evaluate engineered tissue. The magnetic field applied by the MRI to generate an MRI image may generally be between about 10 and 70 MHz. In some examples, the magnetic field frequency used to generate the MRI image may be between about 10 and 50 MHz. In some examples, the magnetic field frequency used to generate the MRI image may be between about 20 and 40 MHz. The frequency used to generate the MRI image in the block 410 may be higher than a magnetic field frequency used to rupture the magnetic nanoparticles included in the engineered tissue. In this manner, the magnetic nanoparticles may not be ruptured during the imaging process.

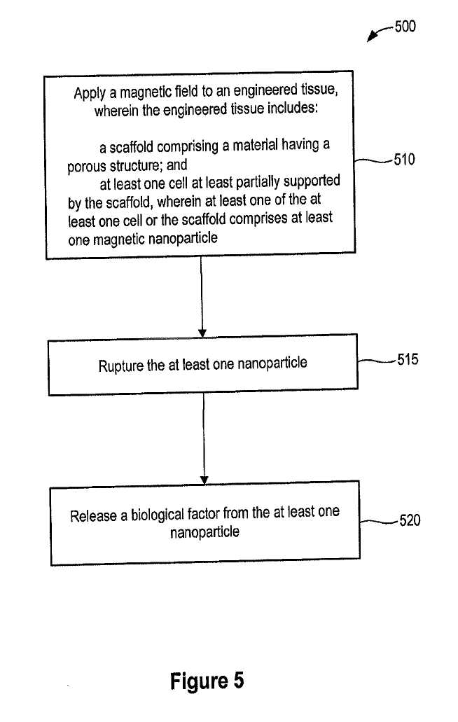

FIG. 5 is a flowchart of a method 500 of controlling tissue growth. In block 510, a magnetic field may be applied to an engineered tissue. As described above, the engineered tissue may include a scaffold. The engineered tissue may further include a cell at least partially supported by the scaffold. At least one of the cell or the scaffold may include magnetic nanoparticles. Examples of suitable engineered tissues have been described above.

Block 510 may be followed by block 515. In block 515, at least one nanoparticle may be ruptured. As has been described above, the magnetic nanoparticles may be ruptured by applying a magnetic field to the magnetic nanoparticles. Suitable frequencies and durations for magnetic field exposure have been described above. A magnetic resonance imaging system may apply the magnetic field to the engineered tissue to rupture at least one magnetic nanoparticle in block 515. Magnetic nanoparticles may be reversibly or irreversibly ruptured in block 515, as has been described above with reference to FIGS. 2A-2C.

In some examples, more than one type of magnetic nanoparticle may be ruptured in the block 515, or using additional steps not shown in FIG. 5. For example, a first magnetic field at a first frequency may be applied to the engineered tissue to rupture a first type of magnetic nanospheres which may, for example, have a particular outer shell thickness. The first frequency, however, may not rupture magnetic nanoparticles having a different particular outer shell thickness. A second magnetic field at a second frequency may be applied to the engineered tissue at a different time to rupture the second type of magnetic nanospheres having a different outer shell thickness. The first and second magnetic nanosphere types may contain different chemicals. In this manner, a chemical to be released may be selected by selecting a frequency of an applied magnetic field.

Block 520 may follow block 515. In block 520, a chemical, such as a biological factor, may be released from the ruptured magnetic nanoparticles. Example chemicals have been described above.

Accordingly, application of a magnetic field may be used to rupture one or more magnetic nanoparticles, delivering a chemical to selected regions of the engineered tissue in the vicinity of the ruptured magnetic nanoparticles. In this manner, growth of the engineered tissue may be controlled by selective release, for example, of growth factor to facilitate or enhance growth or toxin to inhibit or prohibit growth.

In some examples, application of a magnetic field to rupture a magnetic nanoparticle in block 515 may be performed by a same magnetic resonance imaging system, and in some examples in a same MRI session, as the magnetic field to generate an image in block 410 of FIG. 4. In some examples, however, the frequency of the magnetic field applied in the block 515 to rupture magnetic nanoparticles may be different than a frequency of the magnetic field applied in the block 410 to generate an MRI image. Examples of magnetic field frequencies to generate MRI images have been described above and may generally be selected to be less than a frequency for rupturing magnetic nanoparticles of the engineered tissue. In some examples, the magnetic field frequency used to rupture magnetic nanoparticles may be approximately between 10-100 kHz, and in some examples the frequency used to rupture the magnetic nanoparticles may be between 50-100 kHz.

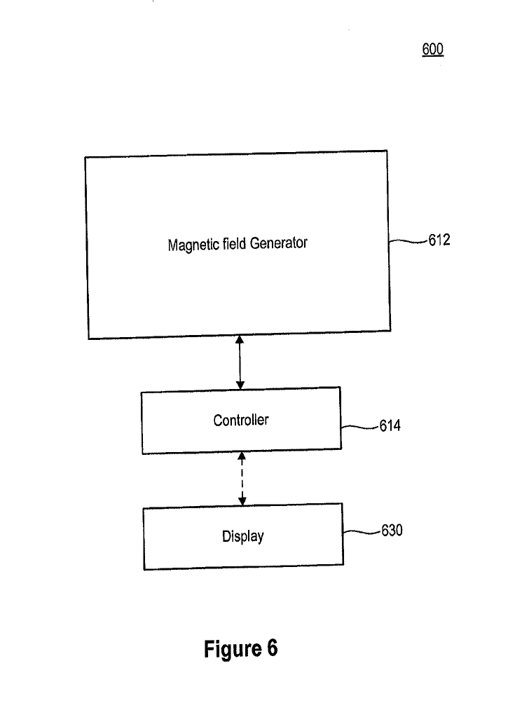

FIG. 6 is a schematic illustration of a magnetic resonance imaging system 600. The magnetic resonance imaging system 600 includes a magnetic field generator 612 coupled to a controller 614, which may be coupled to a display 630.

An engineered tissue, such as the engineered tissue 200 of FIG. 3, may be positioned within a magnetic field generated by the magnetic field generator 612. In some examples, the magnetic field generator 612 forms part of a magnetic resonance imaging machine, and the engineered tissue may be placed in the magnetic resonance imaging machine.

The controller 614 may be configured to generate an image of the engineered tissue, using MRI techniques which have generally been described above. For example, the controller 614 may be configured to provide one or more control signals to the magnetic field generator 612 to generate a magnetic field at a frequency suitable for imaging the engineered tissue. As described above, the controller may be configured for measuring a water diffusion density spectra associated with an engineered tissue, and generating a diffusion spectrum image. Images generated using the controller 614 and magnetic field generator 612 may be displayed on the display 630. The display 630 may be connected to the controller 614 using any communication mechanism, wired or wireless. The display 630 may be located in a same location, such as a same building or room, as the magnetic field generator 612, or may be in a remote location.

The controller 614 may additionally or instead be configured to provide a control signal to the magnetic field generator to generate a magnetic field at a frequency sufficient to rupture one or more nanoparticles of an engineered tissue. Suitable magnetic frequencies have been described above.

The magnetic field generator 612, controller 614, and/or the display 630 may operate together to perform some or all of the methods described above with reference to FIGS. 4 and 5. For example, in block 410 of FIG. 4, a magnetic resonance image may be generated by the controller 614 and magnetic field generator 612 and displayed on the display 630. In block 515 of FIG. 5, at least one nanoparticle may be ruptured using the magnetic field generator 612 and controller 614.

The present disclosure is not to be limited in terms of the particular examples described in this application, which are intended as illustrations of various aspects. Many modifications and examples can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and examples are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular examples only, and is not intended to be limiting.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to examples containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into subranges as discussed above. Finally, as will be understood by one skilled in the art, a range includes each individual member. Thus, for example, a group having 1-3 items refers to groups having 1, 2, or 3 items. Similarly, a group having 1-5 items refers to groups having 1, 2, 3, 4, or 5 items, and so forth.

While the foregoing detailed description has set forth various examples of the devices and/or processes via the use of block diagrams, flowcharts, and/or examples, such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one example, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the examples disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. For example, if a user determines that speed and accuracy are paramount, the user may opt for a mainly hardware and/or firmware vehicle; if flexibility is paramount, the user may opt for a mainly software implementation; or, yet again alternatively, the user may opt for some combination of hardware, software, and/or firmware.

In addition, those skilled in the art will appreciate that the mechanisms of the subject matter described herein are capable of being distributed as a program product in a variety of forms, and that an illustrative example of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While various aspects and examples have been disclosed herein, other aspects and examples will be apparent to those skilled in the art. The various aspects and examples disclosed herein are for purposes of illustration and are not intended to be limiting, with the true scope and spirit being indicated by the following claims.

### Example

The following prophetic example is provided for ease in understanding material described herein. The example is not intended to be limiting.

A series of scaffold layers may be stacked together to simulate a structure of a pancreas. Each layer may have cytokines and/or other cells to simulate the distribution of cells in a typical pancreas. Three sizes of nanoparticles may be incorporated into the scaffold. A first size of nanoparticles may contain agents suitable to stimulate the growth of a first type of cell, and the first size of nanoparticles may be distributed in layers of the scaffold having the first type of cell. A second size of nanoparticles may contain different agents suitable to stimulate the growth of a second type of cell, and the second size of nanoparticles may be distributed in layers of the scaffold having the second type of cell. A third size of nanoparticles may contain an agent suitable to arrest the growth of the first and second types of cells. All layers may contain the third size of nanoparticles.

A magnetic field suitable to rupture the first size of nanoparticles may be applied to release the enclosed agent and stimulate growth of the first type of cells. A magnetic field suitable to rupture the second size of nanoparticles may then be applied to release the enclosed agent and stimulate growth of the second type of cells. When sufficient growth has occurred, as determined by time or observation of the tissue growth, a magnetic field suitable to rupture the third size nanoparticles may be applied to release the enclosed agent and stop cell growth.

1-8. (canceled)

9. An engineered tissue comprising:
a scaffold material;
at least one cell at least partially supported by the scaffold material, wherein the at least one cell comprises at least one magnetic nanoparticle, wherein the at least one magnetic nanoparticle is configured to rupture in response to a magnetic field.

10. The engineered tissue of claim 9, wherein the scaffold material comprises a polymer.

11. The engineered tissue of claim 9, wherein the at least one cell comprises a stem cell.

12. The engineered tissue of claim 9, wherein the magnetic nanoparticle comprises a magnetic shell.

13. The engineered tissue of claim 12, wherein the magnetic shell at least partially contains a biological factor including at least one of a growth factor, an amino acid, a drug, a toxin, or combinations thereof.

14. A method of evaluating engineered tissue, the method comprising:
providing an engineered tissue including:
a scaffold material;
at least one cell at least partially supported by the scaffold material, wherein at least one of the at least one cell or the scaffold comprises at least one magnetic nanoparticle; and
generating a magnetic resonance image of the engineered tissue.

15. The method of claim 14, wherein said generating a magnetic resonance image comprises mapping a diffusion tensor of water associated with the at least one cell.

16. The method of claim 14, wherein said generating a magnetic resonance image comprises measuring a water diffusion density spectra associated with the at least one cell.

17. The method of claim 14, further comprising characterizing at least one of development, connectivity, or differentiation of the engineered tissue based on the magnetic resonance image of the engineered tissue.

18. A method of controlling tissue growth, the method comprising:
applying a magnetic field to an engineered tissue, wherein the engineered tissue includes:
a scaffold material;
at least one cell at least partially supported by the scaffold material, wherein at least one of the at least one cell or the scaffold material comprises at least one magnetic nanoparticle;
rupturing the at least one magnetic nanoparticle; and
releasing a biological factor from the at least one magnetic nanoparticle.

19. The method of controlling tissue growth according to claim 18, wherein said applying a magnetic field comprises using a magnetic resonance imaging system.

20. The method of controlling tissue growth according to claim 18, further comprising generating a magnetic resonance image of the engineered tissue.

21. The method of controlling tissue growth according to claim 20, wherein said applying a magnetic field to an engineered tissue comprises:
applying a first magnetic field to the engineered tissue to generate the magnetic resonance image of the engineered tissue; and
applying a second magnetic field to the engineered tissue to rupture the at least one magnetic nanoparticle.

22. The method of controlling tissue growth according to claim 18 wherein the at least one magnetic nanoparticle comprises a first magnetic nanoparticle and a second magnetic nanoparticle, and wherein said applying a magnetic field to an engineered tissue comprises applying a magnetic field having a strength, frequency, or combination thereof, selected to rupture the first magnetic nanoparticle and not to rupture the second magnetic nanoparticle.

23. The method of controlling tissue growth according to claim 18, wherein the magnetic nanoparticle at least partially contains a biological factor including at least one of a growth factor, an amino acid, a drug, a toxin, or combinations thereof.

24. A magnetic resonance imaging system, comprising:
a magnetic field generator; and
a controller coupled to the magnetic field generator, the controller configured to provide at least one first control signal to the magnetic field generator to generate a first magnetic field having a first frequency configured to generate an image of an engineered tissue and further configured to provide at least one second control signal to the magnetic field generator to generate a second magnetic field having a second frequency configured to rupture at least one magnetic nanoparticle associated with the engineered tissue.

25. The magnetic resonance imaging system of claim 24, wherein said controller is further configured for measuring a water diffusion density spectra associated with the engineered tissue.

26. The magnetic resonance imaging system of claim 24, wherein said second magnetic field has a frequency configured to rupture the at least one magnetic nanoparticle and leave another magnetic nanoparticle intact.

27. The magnetic resonance imaging system of claim 24, further comprising a display coupled to the controller and configured to display a magnetic resonance image of the engineered tissue.
